(19) United States
(12) Patent Application Publication
(10) Pub. No.: US 2012/0283740 A1
(43) Pub. Date: Nov. 8, 2012

(54) SURGICAL HANDPIECE

(75) Inventors: Roland-Alois Hoegerle, Tuttlingen (DE); Uwe Mattes, Tuttlingen (DE); Edgar Blust, Koenigsfeld (DE); Ralf Pfister, Trossingen (DE)

(73) Assignee: Aesculap AG, Tuttlingen (DE)

(21) Appl. No.: 13/457,582

(22) Filed: Apr. 27, 2012

(30) Foreign Application Priority Data
May 6, 2011 (DE) 10 2011 050 191.6

(51) Int. Cl. A61B 17/32; A61B 17/00; A61B 17/88; A61B 17/16; A61B 17/14

(52) U.S. Cl. 606/104; 606/180; 606/176; 606/167; 606/1

(57) ABSTRACT

The present invention relates to a surgical handpiece for driving a surgical tool, with a housing, in which there are arranged a drive in the form of a compressed air motor and at least one bearing or gear part cooperating with the drive on the output side, further comprising at least one lubricant spray connection for coupling to a lubricant spray source, and which is in fluidic connection with at least one of the at least one bearing or gear part and the compressed air motor.

### CROSS-REFERENCE TO RELATED APPLICATIONS

This application claims the benefit of German Patent Application No. 10 2011 050 191.6 filed on May 6, 2011.

The present disclosure relates to the subject matter disclosed in German patent application number 10 2011 050 191.6, filed May 6, 2011, which is incorporated by reference herein in its entirety and for all purposes.

### FIELD OF THE INVENTION

The present invention relates to surgical handpieces for driving a surgical tool generally, and more specifically to a surgical handpiece for driving a surgical tool, with a housing, in which there are arranged a drive in the form of a compressed air motor and at least one bearing or gear part cooperating with the drive on the output side.

### BACKGROUND OF THE INVENTION

Surgical motor systems are known in modular and also integrated construction. In the case of modular construction, the handpiece, which can be coupled to a machining tool for movement thereof, is separate from the drive. The handpiece then comprises at least one bearing or gear part, if necessary. A coupling serves to connect such a handpiece to the drive. In other words, the handpiece and the motor can be separated. As a result, components of the motor system can be cleaned better, since internal cavities thereof are more readily accessible. However, a modular construction also has disadvantages. The coupling between the handpiece and the drive results in a lengthening of the unit formed by the handpiece and the drive. Moreover, the coupling causes faults, i.e. both with respect to the production and to the use of the components.

In contrast hereto, an integrated construction of a surgical handpiece that comprises not only a bearing or gear part, but also the drive provides advantages. Such a handpiece can be configured to be smaller and more compact. Moreover, weight is saved by integrating the drive into the handpiece. Moreover, drive and output shafts can be combined to form a drive train. Since bearing points or play in the region of a coupling between a handpiece comprising only bearing or gear parts and the drive cannot occur because of the integrated construction, the running quietness is additionally improved.

To enable the bearing and gear parts of the handpiece to be permanently usable, the handpiece can either be completely sealed or configured in a so-called open construction. In the case of sealing, all the bearing or gear parts are sealed and provided with a permanent lubrication.

However, the open construction has the disadvantage in particular that bearing and gear parts must be lubricated after every cleaning and before every sterilisation. This is not necessary in the case of a housing sealed for its service life. In the case of a drive in the form of a compressed air motor, this must be sealed on the output side as required by the system. However, a lubrication of a handpiece is conducted from the so-called “clean” side to the so-called “unclean” side. The unclean side of the handpiece forms the end that can be coupled to the tool. The clean side forms the drive side of the motor. Therefore, lubrication of bearing and gear parts on the output side is not possible in the case of a compressed air motor that is sealed to the front or towards the clean side.

Therefore, it would be desirable to provide a surgical handpiece which allows lubricating the compressed air motor and/or further components thereof in a simple and reliable manner.

### SUMMARY OF THE INVENTION

In an aspect of the invention a surgical handpiece for driving a surgical tool, with a housing, in which there are arranged a drive in the form of a compressed air motor and at least one bearing or gear part cooperating with the drive on the output side, further comprises at least one lubricant spray connection for coupling to a lubricant spray source. Said at least one lubricant spray connection is in fluidic connection with at least one of the at least one bearing or gear part and the compressed air motor.

### BRIEF DESCRIPTION OF THE DRAWING FIGURES

The foregoing summary and the following description may be better understood in conjunction with the drawing figures, of which:

FIG. 1 is a schematic view in longitudinal section of a first exemplary embodiment of a surgical handpiece;

FIG. 1A is an enlarged view of section A in FIG. 1;

FIG. 2 is a sectional view similar to FIG. 1 of a further exemplary embodiment of a surgical handpiece;

FIG. 2A is an enlarged view of area B in FIG. 2;

FIG. 3 is a sectional view similar to FIG. 1 of a further exemplary embodiment of a surgical handpiece;

FIG. 4 is a sectional view similar to FIG. 1 of a further exemplary embodiment of a surgical handpiece;

FIG. 4A is a view in the direction of arrow C in FIG. 4;

FIG. 5 is a sectional view similar to FIG. 1 of a further exemplary embodiment of a surgical handpiece;

FIG. 6 is a sectional view similar to FIG. 1 of a further exemplary embodiment of a surgical handpiece;

FIG. 7 is a sectional view similar to FIG. 1 of a further exemplary embodiment of a surgical handpiece;

FIG. 7A is an enlarged view of area D in FIG. 7; and

FIG. 8 is a sectional view similar to FIG. 1 of a further exemplary embodiment of a surgical handpiece.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a surgical handpiece for driving a surgical tool, with a housing, in which there are arranged a drive in the form of a compressed air motor and at least one bearing or gear part cooperating with the drive on the output side, further comprising at least one lubricant spray connection for coupling to a lubricant spray source, and which is in fluidic connection with at least one of the at least one bearing or gear part and the compressed air motor.

To further develop a surgical handpiece of the above-described type in such a manner has the advantage in particular that by providing at least one lubricant spray connection the compressed air motor and/or the at least one bearing or gear part can be subjected to a lubricant spray in a simple manner for lubrication thereof. For this, it is merely necessary, for example, to couple the desired lubricant spray source to the lubricant spray connection of the handpiece. Thus, also in the case of a surgical handpiece configured in integrated construction and thus comprising a drive that cannot be disconnected, a suitable lubricant spray with a lubricant, e.g. an oil, contained therein can be directed precisely to where it is needed, namely to the at least one bearing or gear part and/or to the compressed air motor. An optimum cleaning capacity with a significantly improved lubricating capacity of components to be lubricated in comparison to conventional handpieces can thus be achieved. It is conceivable to provide two, three or more lubricant spray connections on the handpiece. It is favourable, however, if only a single lubricant spray connection is provided. In particular, this allows in principle the lubrication of the handpiece components to be lubricated by means of only a single lubricant spray burst.

It is favourable if at least one lubricant spray connection conduit is provided for forming a fluidic connection between the at least one lubricant spray connection and the at least one bearing or gear part. Thus, a lubricant spray can pass through the lubricant spray connection conduit directly from the lubricant spray connection to the at least one bearing or gear part or also to the compressed air motor in order to lubricate the said components.

It is advantageous if the housing comprises at least one seating for the at least one bearing or gear part, in which seating the at least one bearing or gear part is arranged, and if the at least one lubricant spray connection is in fluidic connection with the at least one seating. To configure the housing in such a manner and to arrange or configure the at least one lubricant spray connection in such a manner enables a lubricant to be directed from the lubricant spray connection to the at least one bearing or gear part in a simple and reliable manner.

Advantageously, the at least one lubricant spray connection conduit connects the at least one lubricant spray connection and the at least one seating to one another to form a fluidic connection. In this way, the desired fluidic connection can be created in a particularly simple and reliable manner.

The handpiece can be produced in a particularly simple manner if the at least one lubricant spray connection conduit is configured in the form of a flexible tube- or pipe-type connection conduit or in the form of a connection channel arranged or configured on the housing. Depending on the size of the handpiece, the at least one lubricant spray connection conduit can thus be configured by a separate structural part or by a corresponding shaping of parts forming the housing in cooperation with components arranged in the housing. Two, three or more lubricant spray connection conduits can, of course, also be provided for the fluidic connection of one or more lubricant spray connections to all the components of the handpiece to be lubricated.

The housing advantageously comprises a drive seating, in which the drive is arranged. The drive can thus be mounted on the handpiece in a defined manner.

The at least one seating is preferably arranged or configured on the output side of the drive seating. This allows, for example, a gear to be arranged or configured between the drive and a tool that can be coupled to a distal end of the handpiece.

In order to prevent a loss of compressed air at the handpiece, it is advantageous if the drive is arranged in the housing to be sealed in an airtight manner on the output side.

The ease of handling of the handpiece in particular during lubrication thereof can be improved in particular by the handpiece comprising only a single lubricant spray connection. For example, lubricant can thus be introduced into the handpiece in the form of a lubricant spray with only one or also two or more spray bursts to lubricate the components that need lubrication.

The handpiece can be configured in a particularly compact manner if the at least one lubricant spray connection is arranged or configured in a housing wall of the housing and/or to project from the housing wall. This enables the lubricant spray connection to be integrated into the handpiece in a simple manner.

To improve an accessibility of the at least one bearing or gear part when this is arranged or configured on the handpiece on the output side in relation to the drive, it is favourable if the at least one lubricant spray connection is arranged or configured on the output side of the drive. In this way, the lubricant can be directed along the shortest possible path from the lubricant spray connection to the at least one bearing or gear part.

The handling of the handpiece can be improved in a simple manner in particular by the at least one lubricant spray connection being arranged or configured on the proximal side of the drive, and the at least one lubricant spray connection conduit being arranged or configured laterally past the drive. This configuration allows the handpiece to be lubricated, e.g. from a proximal end thereof, by subjecting it to a lubricant spray, for example, wherein the proposed arrangement of the lubricant spray connection conduit also allows a lubrication of bearing or gear parts arranged on the distal or output side in a simple or reliable manner.

The accessibility of the lubricant spray connection can be improved in a simple manner by the at least one lubricant spray connection being arranged or configured on a proximal end of the handpiece. A lubrication of the handpiece can thus be conducted from the clean side to the unclean side in a simple manner as described above.

To permit as slender a construction of the handpiece as possible, it is favourable if the at least one lubricant spray connection is arranged or configured to face in the proximal direction.

According to a further preferred embodiment of the invention it can be provided that the at least one lubricant spray connection comprises a self-closing inlet valve. It can thus be ensured that the at least one lubricant spray connection is closed when it is not being subjected to a lubricant spray that is under a minimum pressure in order to open the inlet valve. Thus contaminants can be prevented from passing through the lubricant spray into the interior of the handpiece and, in reverse, lubricant can be prevented from discharging.

To be able to operate the compressed air motor in a simple and reliable manner, it is advantageous if the handpiece comprises at least one compressed air inlet and at least one compressed air outlet, which are in fluidic connection to the compressed air motor. By means of the at least one compressed air inlet the compressed air motor can be subjected to compressed air, which can then flow out of the handpiece again through the at least one compressed air outlet. Two, three, four or even more respective compressed air inlets or outlets are conceivable.

A particularly simple and compact construction of the handpiece is possible in particular if the at least one compressed air inlet forms the at least one lubricant spray connection. In other words, the lubricant can be directed into the interior of the handpiece directly through the at least one compressed air inlet.

In addition, it can be advantageous if the at least one compressed air outlet forms the at least one lubricant spray connection. In this way, a lubricant can be directed into the interior of the handpiece in particular through the compressed air outlet, e.g. in order to lubricate the compressed air motor. A valve can also be optionally provided, which in an operating position separates the compressed air outlet from the lubricant spray connection conduit and only opens in a lubricating position when subjected to pressure, e.g. on introduction of a lubricant spray, in order to thus also lubricate the at least one bearing or gear part.

According to a further preferred embodiment of the invention it can be provided that the compressed air motor comprises a rotor shaft, and that the rotor shaft comprises the at least one lubricant spray connection conduit. This configuration allows a lubricant to be directed through the rotor shaft to a bearing or gear part arranged on the output side.

The rotor shaft is preferably configured in the form of a hollow shaft, which defines a channel. This allows the lubricant to be directed through the rotor shaft directly.

The surgical handpiece can be configured in a particularly compact manner if the channel forms the at least one lubricant spray connection conduit. In other words, the rotor shaft can thus itself form the lubricant spray connection conduit.

Moreover, it can be favourable if the at least one lubricant spray connection conduit is arranged in the channel and is configured to be fixed relative to the housing. In this way, it is possible to prevent the rotor shaft from being subjected to a lubricant internally that should preferably be directed to the at least one bearing or gear part directly.

The handpiece, and in particular its compressed air connections, can be configured in a particularly compact manner if the at least one compressed air inlet is arranged or configured coaxially to the rotor shaft of the compressed air motor.

In addition, it can be advantageous if the at least one compressed air outlet is arranged or configured coaxially to the rotor shaft of the compressed air motor. A coaxial configuration of a compressed air inlet or a compressed air outlet means in particular that only a single inlet or outlet is sufficient on the handpiece.

According to a further preferred embodiment of the invention it can be provided that a closing element impermeable to compressed air in a closed position is arranged or configured in or on the at least one lubricant spray connection conduit. In particular, the lubricant spray connection conduit can be closed with the closing element when the handpiece is in use, i.e. in particular the drive is subjected to compressed air. Compressed air can thus be prevented from escaping through the lubricant spray connection conduit in an unwanted manner.

The handling of the surgical handpiece becomes particularly simple if the closing element is configured in the form of a self-closing valve, which is openable in a lubricating position. For example, it can be arranged and configured in such a manner that it opens automatically when a lubricant spray is directed into the handpiece through the lubricant spray connection, e.g. when the pressure arising in the lubricant spray is high enough that the valve opens automatically. The lubricant can then flow through the lubricant spray connection conduit and reach components of the handpiece to be lubricated.

To prevent contaminants from being able to pass into the interior of the handpiece or compressed air that is provided for driving the compressed air motor from being able to escape in an unwanted manner, it is advantageous if the handpiece comprises at least one lubricant spray connection closure for closing the at least one lubricant spray connection.

The at least one lubricant spray connection closure can be configured in a particularly simple manner if it is arranged or configured on a compressed air coupling, which can be coupled to the handpiece. This means that, for example, when the handpiece is coupled to the compressed air coupling, which is itself connected to a compressed air source, the lubricant spray connection is automatically closed by the lubricant spray connection closure. An operator thus closes the lubricant spray connection automatically by bringing the handpiece into engagement with a compressed air coupling configured accordingly for this.

Moreover, it can be favourable if the handpiece has a self-closing exhaust air duct closing valve, which is closed in an operating position and is open in a lubricating position for forming a fluidic connection between an exhaust air duct connecting to the at least one compressed air outlet and the compressed air motor and the at least one lubricant spray connection conduit. The exhaust air closing valve thus allows in particular a fluidic connection to be created or not between the at least one compressed air outlet and the lubricant spray connection conduit. Such a fluidic connection is desirable for lubricating the handpiece, but not for operating the handpiece with compressed air.

In addition, it can be favourable if the handpiece comprises a compressed air supply interruption arrangement, which is open in an operating position to form a fluidic connection between an air inlet duct connected to the at least one compressed air inlet and the compressed air motor and which is closed in a lubricating position to interrupt the fluidic connection between the at least one compressed air inlet and the air inlet duct. The compressed air supply interruption arrangement thus allows a fluidic connection between the at least one compressed air inlet and the air inlet duct to be selectively interrupted. As a result of this, for example, lubricant cannot pass through the air inlet duct to the compressed air motor if this is not desired.

To be able to subject the at least one bearing or gear part to lubricant in a simple and reliable manner, it is advantageous if the at least one lubricant spray connection conduit is open in the lubricating position.

It is particularly advantageous if the compressed air supply interruption arrangement comprises a closing element movably held on the housing or a valve or a part of a compressed air coupling that is couplable to the handpiece. In this way, a fluidic connection in particular between the at least one compressed air inlet and the air inlet duct can be interrupted by a correspondingly configured valve in a simple manner, i.e. manually operated by an operator or automatically, for example.

The closing element is preferably configured to be manually operable. This allows an operator to open and close the fluidic connection between the compressed air inlet and the air inlet duct in the desired manner.

According to a preferred embodiment of the invention it can be provided that the at least one bearing or gear part is configured in the form of a ball bearing, a sliding bearing, a needle bearing, a bearing shaft or a gear wheel. Two, three or more gear parts can, of course, also be provided on the handpiece that can be lubricated by subjecting them to a lubricant spray.

To be able to perform desired surgical procedures with the handpiece it is advantageous if it comprises a surgical tool coupled to the handpiece on the distal side. The tool can be configured to be selectively detachably connectable to the handpiece or also be connected to be permanently fixed thereto.

The tool is preferably a drilling, milling, sawing, screwing or cutting tool. Such a tool allows a multiplicity of surgical procedures to be performed in the desired manner.

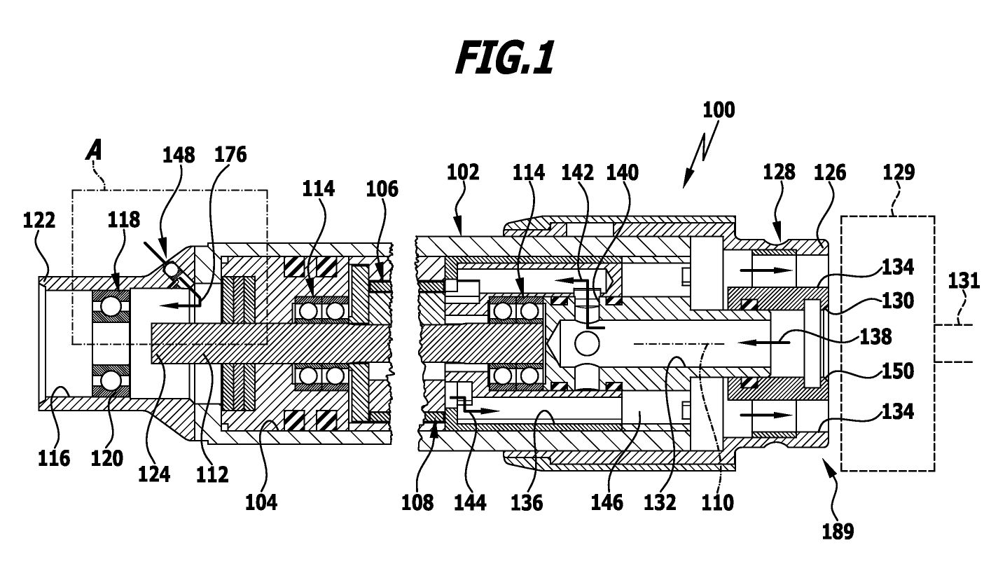

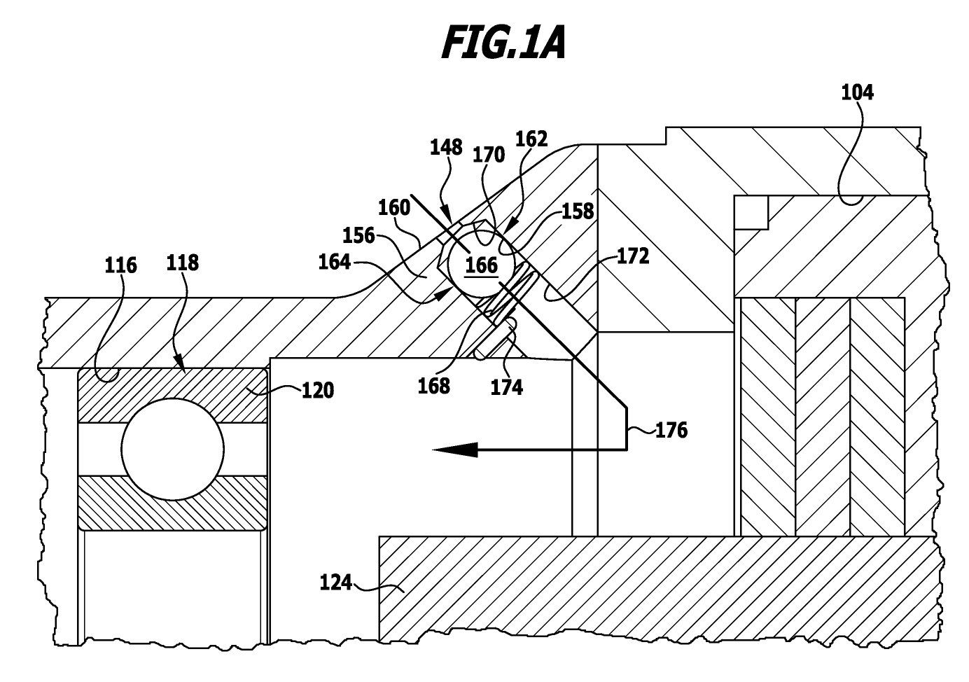

FIG. 1 shows a first exemplary embodiment of a surgical handpiece schematically in longitudinal section given the overall reference 100. It comprises a housing 102 with a recess in the form of a drive seating 104, in which a drive 106 in the form of a compressed air motor 108 is arranged.

The handpiece 100 defines a longitudinal axis 110, which at the same time forms the longitudinal axis of the rotor shaft 112 of the compressed air motor 108. The rotor shaft 112 is mounted at its proximal end on the housing 102 on the output side of the drive 106 respectively with a ball bearing 114. Moreover, the drive 106 is arranged on the distal or output side to be fluid-tight in the housing 102. The rotor shaft 112 projects into a seating 116 for a bearing or gear part 118, which can be configured, for example, in the form of a ball bearing 120 shown schematically in FIG. 1. A distal end 122 of the housing 102 is open so that a proximal end of a machining tool that is not shown in the figures can be inserted into the seating 116 and coupled to a distal end 124 of the rotor shaft 112. A shank of the tool can then be supported on the housing 102 by means of the ball bearing 120, for example, and be oriented coaxially to the longitudinal axis 110 by the ball bearing 120.

A proximal end 126 of the housing 102 is preferably configured in the form of a coupling piece 128 for detachable connection, i.e. temporary coupling, to a tube coupling, which is configured on a end of a connection tube that is not shown in the Figures for connecting the handpiece 100 to a compressed air supply or compressed air source (not shown). The coupling piece 128 has a compressed air inlet 130 configured coaxially to the longitudinal axis 110 and connecting on the inlet side by a fluidic connection to the compressed air motor 108 by means of an air inlet duct 132. A plurality of compressed air outlets 134, which open in proximal direction and are circular in cross-section, are spaced radially from the longitudinal axis 110 on the coupling piece 128 and are configured to surround the compressed air inlet 130 and connect on the exhaust air side by fluidic connection to the compressed air motor 108 by means of an exhaust air duct 136.

To drive the compressed air motor 108 the compressed air inlet 130 is subjected to compressed air, which flows into the handpiece 100 coaxially to the longitudinal axis 110 in the direction of the arrow 138 and after flowing through a cross channel 140 configured on the proximal side of the compressed air motor 108 is finally directed to the compressed air motor 108 in distal direction parallel to the longitudinal axis 110. A flow path of the compressed air through the cross channel 140 is indicated schematically by the arrow 142. On the exhaust air side, the compressed air flows in the direction of the flow path symbolised by the arrow 144 in proximal direction back through the exhaust air duct 136, which connects to a ring space 146 by a fluidic connection, which concentrically surrounds the air inlet duct 132. The ring space 146 itself connects to the compressed air outlets 134 by a fluidic connection.

To lubricate the ball bearing 120, a lubricant spray connection 144 is provided that is configured in a wall 156 of the housing 102 on the output side of the compressed air motor 108. It comprises a hole 158, which is configured to face away from the longitudinal axis 110 and in distal direction inclined about 45° in relation to the longitudinal axis 110. An angle of inclination between the hole 158 and the longitudinal axis 110 can lie selectively in an angular range of 0° to 180°. The hole 158 tapers towards an outer surface 160 of the wall 156. A closing element 162 in the form of a self-closing valve 164 is arranged therein. It comprises a ball 166 that is pressed against a conical valve seat 170 by a coil spring 168. The coil spring 168 is supported against the ball on one side and against a projection 174 projecting from an inside wall 172 of the hole 158 on the other side.

If a coupling piece of a lubricant spray source, for example, is attached to the housing 102 from the outside, pressurised lubricant spray can move the balls 166 into the hole 158 against the action of the coil spring 158 so that the valve 164 can be opened and lubricant can flow through the hole 158 substantially along a flow path represented schematically by the arrow 176 into the seating 116 to lubricate the bearing or gear part 118.

Moreover, the compressed air inlet 130 can also form a lubricant connection 150 to lubricate the compressed air motor 108 after cleaning and before sterilisation by appropriate connection of a lubricant spray source. The lubricant spray then follows the flow path described above.

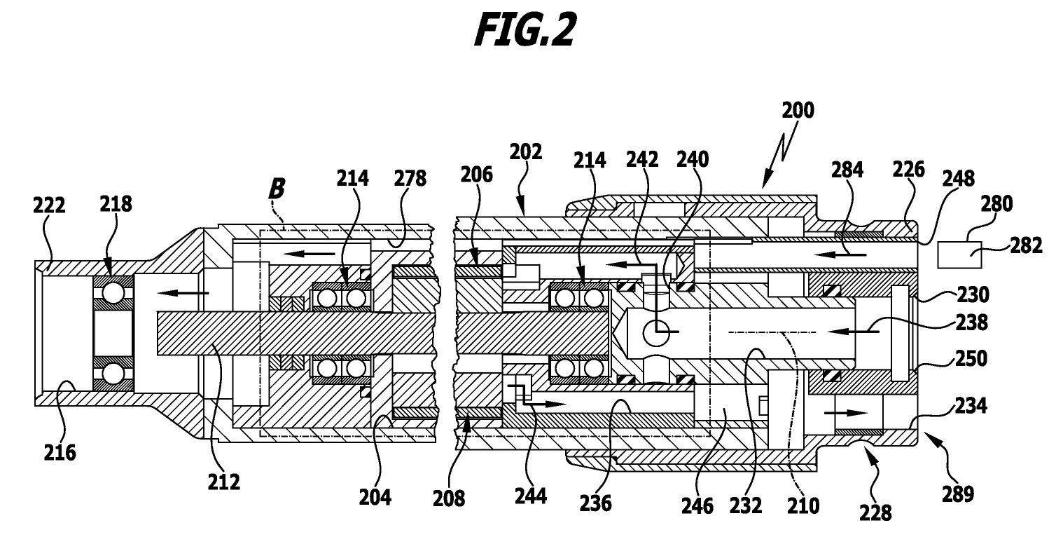

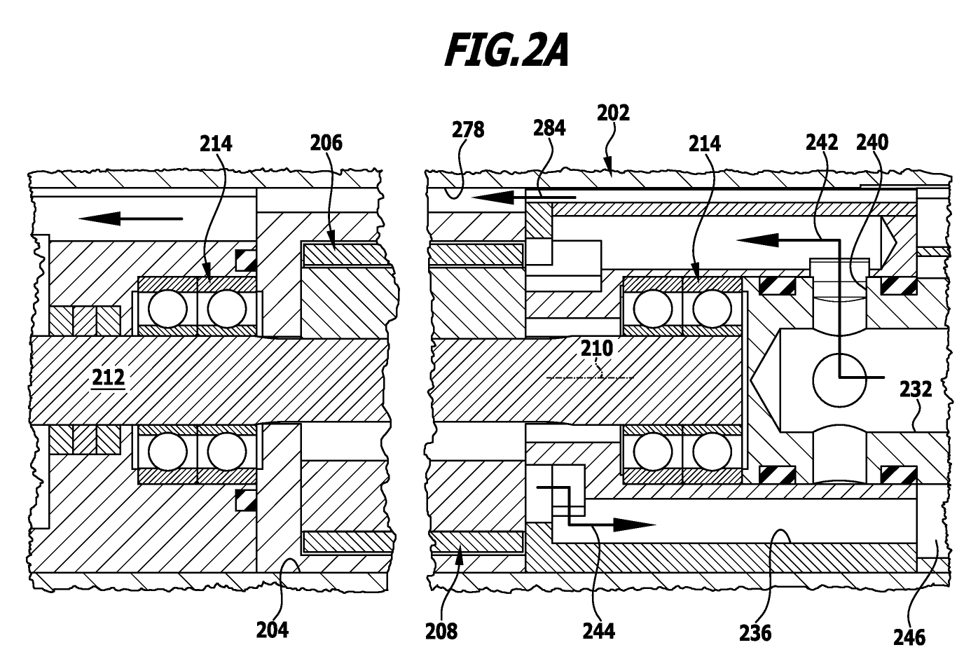

FIG. 2 schematically shows a further exemplary embodiment of a surgical handpiece given the overall reference 200. The last two digits of the references for structural parts and components of the handpiece 200 that are the same as those of handpiece 100 are identical. Moreover, this also applies to all exemplary embodiments described in more detail below.

The construction of the handpiece 200 differs from handpiece 100 substantially in that the lubricant connection 248 is not provided on the output side of the drive 206, but on the end 226 facing in proximal direction. A lubricant spray connection conduit 278, which runs eccentrically to the longitudinal axis 210, but substantially parallel thereto, laterally past the drive 206 and fluidically connects the lubricant spray connection 248 and the seating 216, serves to create a fluidic connection between the lubricant spray connection 248 and the seating 216. If a lubricant spray source 180, which is shown schematically in FIG. 2 and can be configured in the form of a spray can 282, for example, is coupled to the lubricant spray connection 248, then the lubricant spray can flow in the direction of arrow 284 into the lubricant spray connection conduit 278 and laterally past the drive into the seating 216 and lubricate the ball bearing 220. The compressed air inlet 230 can be used as lubricant spray connection 250 for lubrication of the compressed air motor 208, i.e. in the above-described manner.

Thus, the handpiece 200 enables a simple lubrication of the bearing or gear part 118 from the clean side of the handpiece 200 towards the unclean side, i.e. to the distal end 222, which can be coupled to a machining tool.

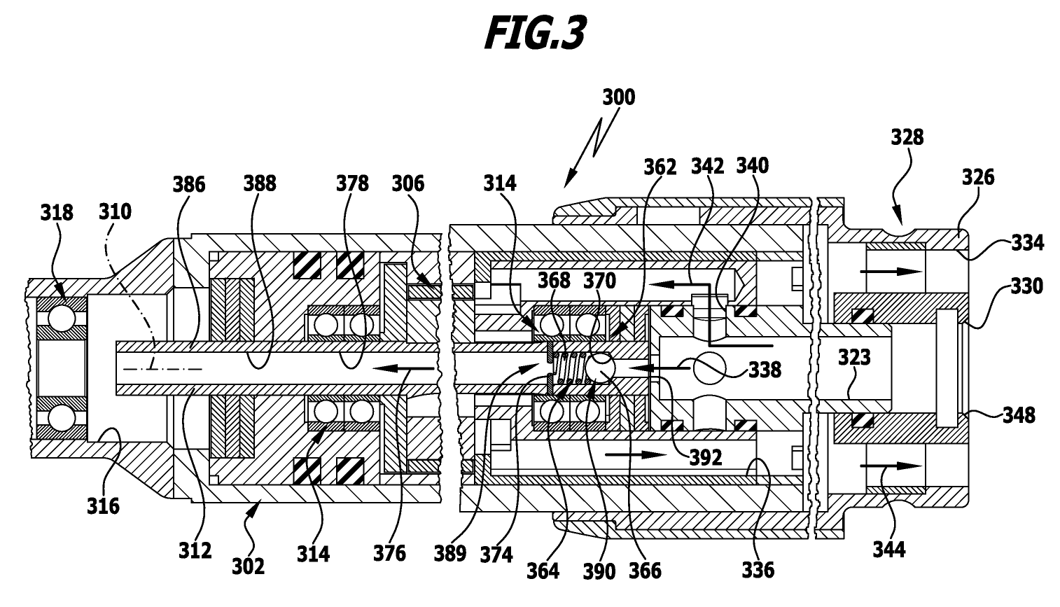

A further exemplary embodiment of a surgical handpiece is shown schematically in FIG. 3 and given the overall reference 300. As already indicated in the case of handpiece 200, structural parts and components of handpiece 300 that are identical to structural parts and components of the handpiece 100 are given references with the same last two digits.

The construction of the handpiece 300 substantially corresponds to that of handpiece 100. However, it differs in particular in that the rotor shaft 312 is configured in the form of a hollow shaft 386, which in its interior defines a channel 388 that forms a lubricant spray connection conduit 378. On the proximal side the channel 388 is closed by a compressed air supply interruption arrangement 389, which comprises a closing element 390 in the form of a valve 364. The valve 364 comprises a ball 366, which is biased by a coil spring 368 that is supported against an annular projection 374 of the channel 388 on the distal side and against the ball 366 on the other side, so that this is pressed against a hollow spherical valve seat 370 facing in distal direction in order to close the channel 388 in a closing position shown in FIG. 3.

In the case of handpiece 300 the compressed air inlet 330 forms the single lubricant spray connection 348. A connection pipe of a lubricant spray source can be coupled through this connection to an inlet 392 of the channel 388, so that the valve 364 opens under the action of pressure upon introduction of a lubricant spray and lubricant can flow into the seating 316 coaxially to the longitudinal axis 310 in the direction of the arrow 376 for lubrication of the ball bearing 320. Because of its construction the valve 364 is configured to open automatically if it is not opened by a corresponding lubricant spray pressure. The valve 364 is preferably configured so that upon operation of the handpiece 300, i.e. on subjecting the compressed air motor 308 to compressed air, it is it still closed, i.e. closes the lubricant spray connection conduit 378 to be impermeable to compressed air in the closing position shown in FIG. 3. If valve 364 is open, it assumes a lubricating position. As in the case of handpieces 100 and 200, the compressed air motor 308 is also lubricated by introducing a lubricant spray through the compressed air inlet 330.

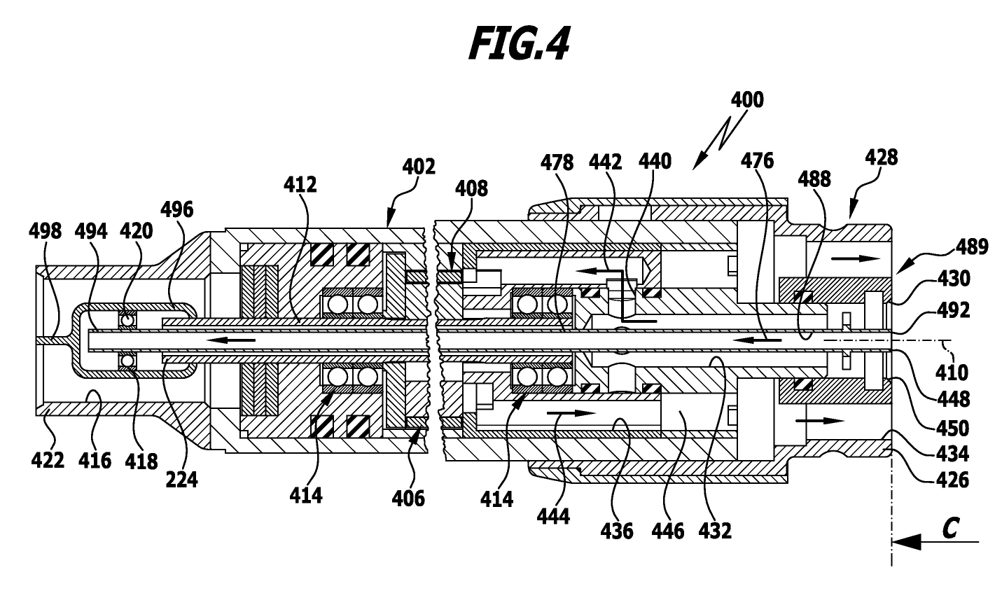

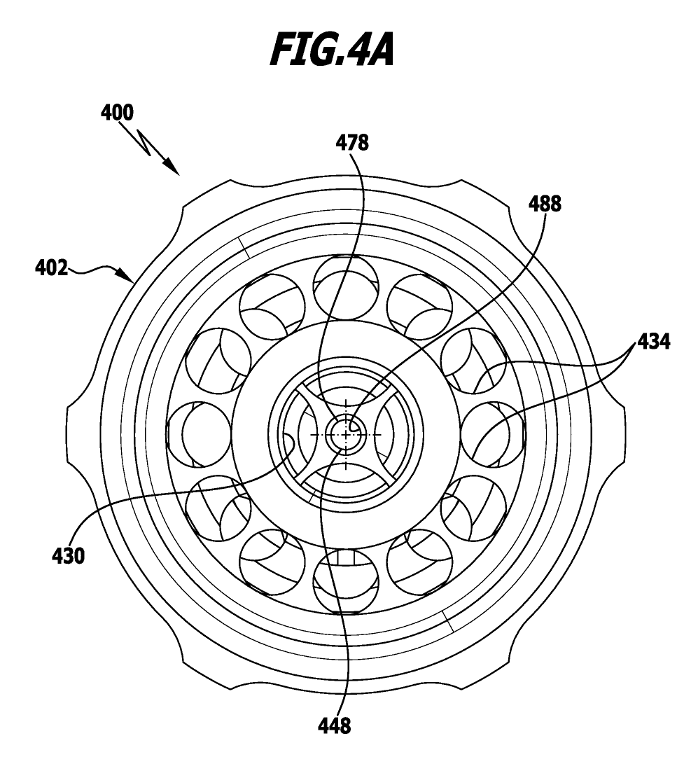

A further exemplary embodiment of a surgical handpiece is shown schematically in FIG. 4 and given the overall reference 400. It is similar in construction to handpiece 300.

Handpiece 400 also has a rotor shaft 412, which defines a channel 488 extending coaxially to the longitudinal axis 410. Arranged in the channel 488 is a tubular lubricant spray connection conduit 478, which extends as far as the proximal end 426 of the handpiece 400, but does not touch the rotor shaft 412 and is arranged to be fixed relative to the housing 402. In the region of a distal end 494 of the lubricant spray connection conduit 478 this bears on the outside a bearing or gear part 418 in the form of a ball bearing 420. The ball bearing 420 is supported on a coupling sleeve 496 radially on the outside that engages over a distal end 424 of the rotor shaft 412 on the proximal side and is connected to this to be fixed against rotation. The coupling sleeve 496 comprises a coupling projection 498 oriented in distal direction that can be brought into engagement with a tool in order to set this in rotation. A proximal end of the lubricant spray connection conduit 478 defines an inlet 492 in the form of a lubricant spray connection 448. This is surrounded coaxially by a compressed air inlet 430.

For lubricating the ball bearing 420 a lubricant spray source is coupled to the lubricant spray connection 448 and the lubricant spray is directed through the lubricant spray connection conduit 478 passing through the channel 488 coaxially to the longitudinal axis 410 in the direction of arrow 476 for lubricating the ball bearing 420. The compressed air motor 408 can be lubricated with lubricant spray that is introduced through the compressed air inlet 430, which in turn forms a lubricant spray connection 450.

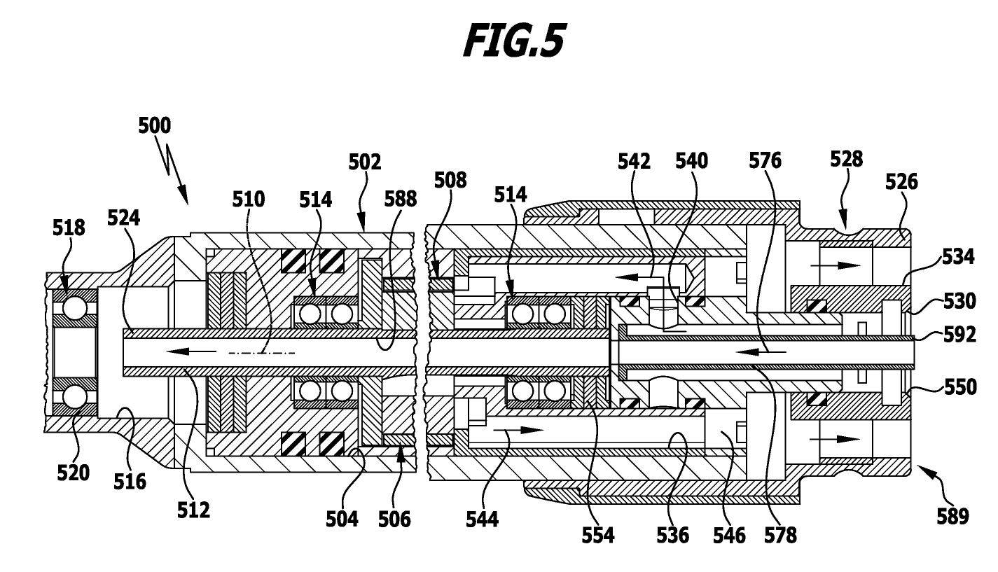

A further exemplary embodiment of a surgical handpiece is shown schematically in FIG. 5 and given the overall reference 500. It is similar in construction to the surgical handpiece 400.

A tubular lubricant spray connection conduit 578 extends from a proximal end 526 coaxially to the longitudinal axis 510 to close to a proximal end of the rotor shaft 512. The lubricant spray connection conduit 578 is configured to be fixed relative to the housing 502. It is in turn coaxially surrounded by a compressed air inlet 530 that forms a lubricant spray connection 550. On the proximal side, i.e. in a region of its proximal end, the rotor shaft 512 is surrounded by annular sealing elements 554 that seal the rotor shaft 512 fluid-tight in relation to the lubricant spray connection conduit 578.

A ball bearing 520 is arranged in the seating 516, as in the case of handpiece 100. For the lubrication thereof, a lubricant spray can be introduced through an inlet 592 of the lubricant spray connection conduit 578 facing in proximal direction and introduced in the direction of arrow 576 through the lubricant spray connection conduit 578 into the channel 588 of the shaft-shaped rotor shaft 512 and through this as far as the seating 516 in order to lubricate the ball bearing 520. The lubricant spray connection 550 is used for lubrication of the compressed air motor 508.

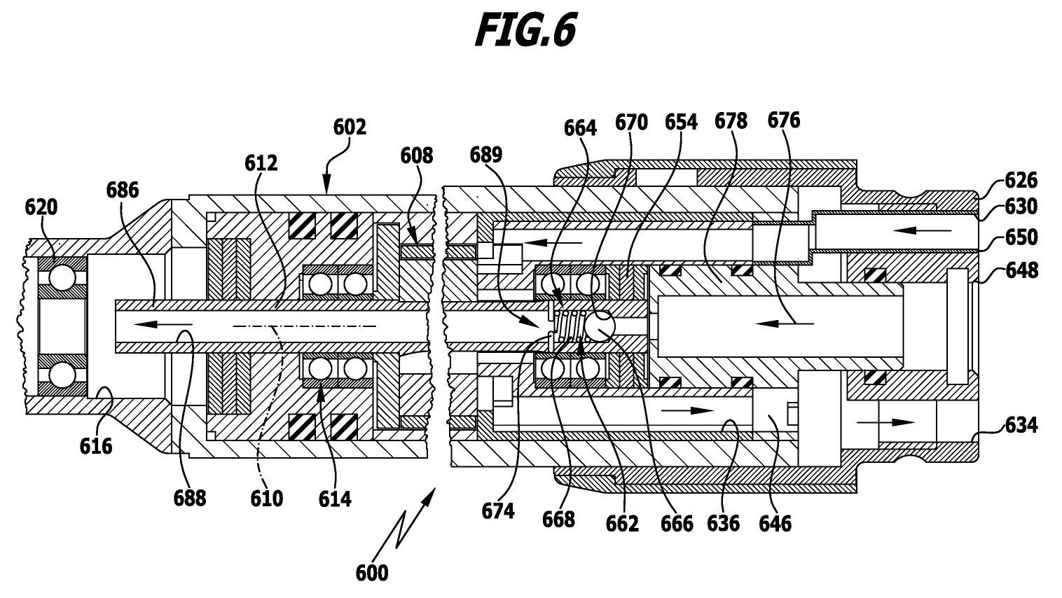

A further exemplary embodiment of a surgical handpiece is shown schematically in FIG. 6 and given the overall reference 600. It has some similarity with the handpiece 500, but differs from this in particular in that the compressed air inlet 630 is configured on the proximal end 626 laterally offset relative to the longitudinal axis 610.

A plurality of compressed air outlets 634 that are coupled to the ring space 646 are also offset laterally to the longitudinal axis 610. Coaxially to the longitudinal axis 610 a lubricant spray connection 648, which is in fluidic connection with a lubricant spray connection conduit 678 that is fixed relative to the housing 602, is configured to face in proximal direction. As in the case of handpiece 500, the rotor shaft 612 is configured in the form of a shaft 686 and is sealed on the proximal side relative to the lubricant spray connection conduit 678 by means of sealing elements 654. The channel 688 forms almost an extension of the lubricant spray connection conduit 678, so that in this, as in the case of handpiece 300, a closing element 662 in the form of a valve 664 is arranged in the rotor shaft 612 to automatically close the channel 688. The valve comprises a ball 666, which is supported against an annular projection 674 of the channel 688 arranged on the distal side, is pressed against a valve seat 670 in proximal direction in order to close the channel 688 in the closing position shown in FIG. 6.

If the lubricant spray connection 648 is coupled to a lubricant spray source, the valve 664 opens because of the acting pressure and allows a lubricant spray to flow in through the lubricant spray connection conduit 678 coaxially to the longitudinal axis in the direction of the arrow 676 through the valve 664 opened in the lubricating position and also through the channel 688 into the seating 616 for lubrication of the ball bearing 620. The compressed air inlet 630 can be used as lubricant spray connection 650 for lubrication of the compressed air motor 608.

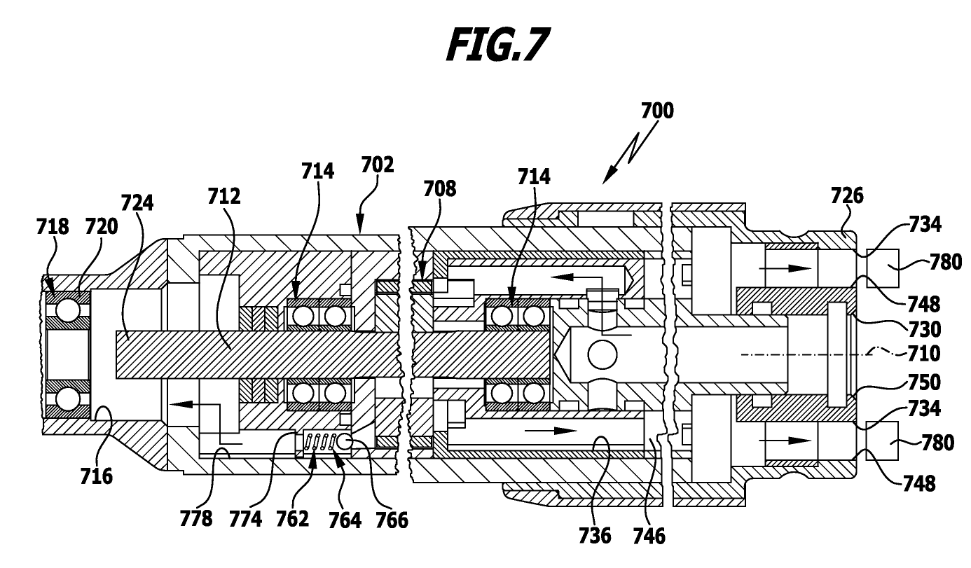

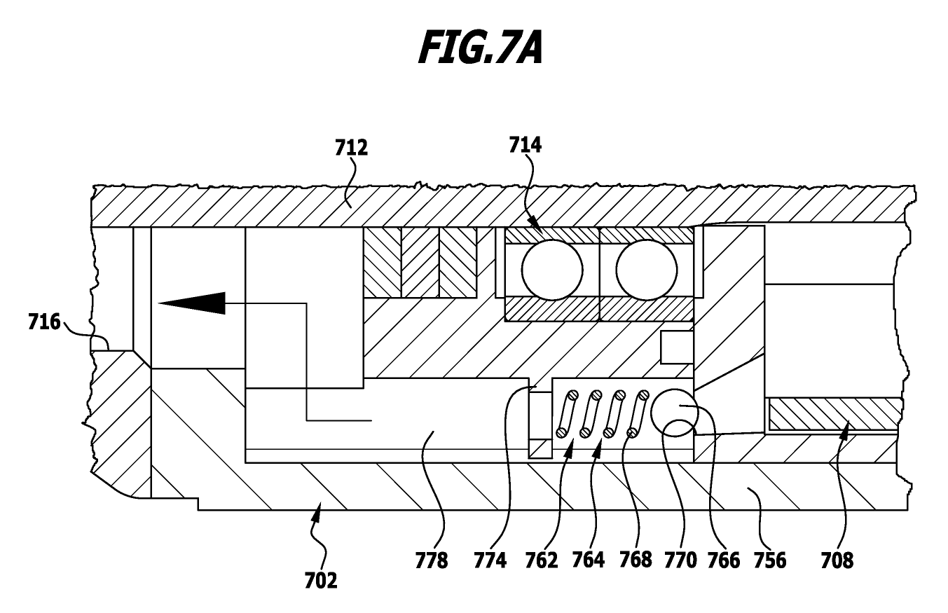

A further exemplary embodiment of a surgical handpiece is shown schematically in FIG. 7 and given the overall reference 700. It is the most similar in construction to handpiece 100. However, it differs from handpiece 100 in that the compressed air outlets 734 can be used as lubricant spray connections 748.

On the output side of the compressed air motor 708 a channel-like lubricant spray connection conduit 778 is formed parallel to the longitudinal axis 710 and close to an outside wall 756 of the housing 702 to create a fluidic connection between the compressed air outlets 734 and the seating 716. A closing element 762 in the form of a valve 764 is arranged in the lubricant spray connection conduit 778. The valve 764 comprises a ball 766, which by means of a coil spring 768, which is supported on the distal side on an annular projection 774 that projects from an inside wall of the lubricant spray connection conduit 778, is pressed in proximal direction against a valve seat 770 and holds the lubricant spray connection conduit 778 closed in an operating position of the handpiece 700. Thus, contaminants can be prevented from being able to pass from the proximal end 726 through the lubricant spray connection conduit 778 into the seating 716. The valve 764 is dimensioned such that the exhaust air exiting from the compressed air motor 708 is not sufficient to open the valve 764.

Alternatively, the surgical handpiece 700 can also be lubricated in such a manner that a lubricant spray is introduced through the compressed air inlet 730, which then forms a lubricant spray connection 750. If the compressed air outlets 734 are closed during this procedure, the valve 764 is also opened by the thus resulting pressure build-up and lubricant spray can pass through the lubricant spray connection conduit 778 into the seating 716. If the compressed air outlets 734 are opened when the handpiece 700 is being sprayed through the lubricant spray connection 750, the compressed air motor 708 is lubricated in the usual manner.

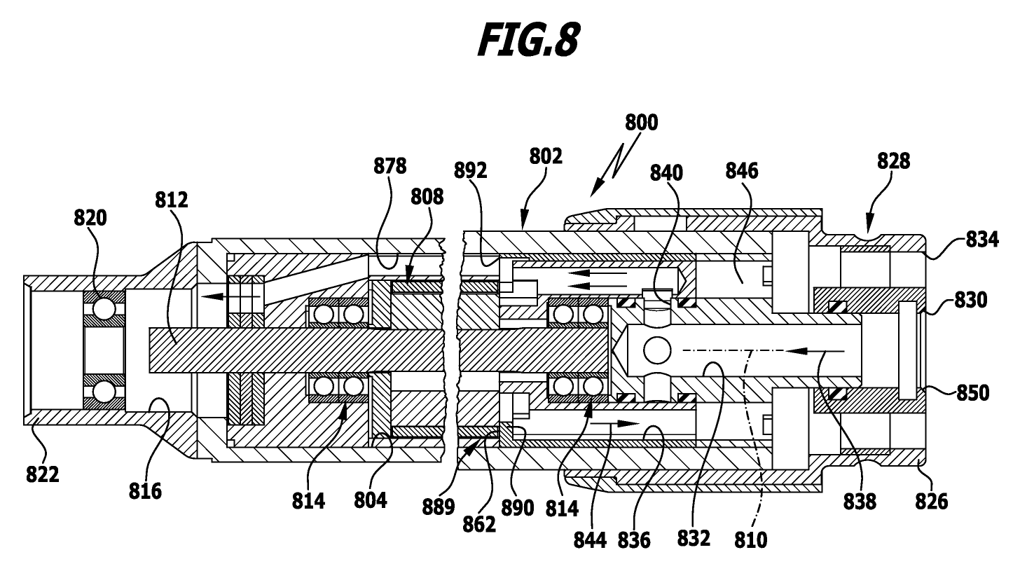

A further exemplary embodiment of a surgical handpiece is shown schematically in FIG. 8 and given the overall reference 800. It is similar in construction to handpiece 700.

A compressed air inlet 830 that serves as lubricant spray connection 850 is configured coaxially to the longitudinal axis 810. An air inlet duct 832 is connected to a supply line to the compressed air motor 808 by means of a cross channel 840. The supply line in turn connects directly by a fluidic connection to a channel-like lubricant spray connection conduit 878 directed laterally eccentrically past the compressed air motor 808. An inlet 892 of the lubricant spray connection conduit 878 is closable by a closing element 862, which is mounted on the housing 802 to be rotatable around the longitudinal axis 810. It can be changed from a closing position, in which the inlet 892 is closed by the closing element 862, into a lubricating position, in which the closing element 862 releases the inlet 892 to create a fluidic connection between the air inlet duct 832 and the lubricant spray connection conduit 878. The lubricating position is shown schematically in FIG. 8. A lubricant spray introduced through the lubricant spray connection 850 can thus, on the one hand, flow through the compressed air motor 808, but, on the other hand, can also flow through the lubricant spray connection conduit 878 past the compressed air motor 808 into the seating 816 to lubricate the ball bearing 820.

A further variant of a surgical handpiece, although not shown in the figures, should however be explained briefly on the basis of handpiece 300. If the valve 364 in handpiece 300 is configured to act in contrary direction in the rotor shaft 312, the handpiece 300 can also be lubricated from the unclean side, i.e. directly by introducing a lubricant spray from the distal direction into the seating 316. In this case, the valve can open and the lubricant spray can flow into the air inlet duct 332 in proximal direction. For example, if the compressed inlet 330 were closed in this case, then the compressed air motor 308 connecting to the seating 316 by fluidic connection can also be lubricated from the distal direction.

What almost all the above-described handpieces have in common is that they allow lubrication of both the compressed air motor or its compressed air cell and of the handpiece, moreover, with its bearing and gear parts with a single spray burst. This thus makes possible an open integrated construction with a compressed air motor that, on the one hand, allows an optimum cleaning of the handpiece, but on the other hand, as a result of the proposed further developments also allows a simple and reliable lubrication of all the relevant components of the handpieces. In particular, a machine conditioning with or without subsequent flushing is possible. For this, the respective lubricant spray connection of the handpiece can be optionally connected to a cleaning fluid source. This can be achieved by means of a suitable coupling piece to a cleaning fluid conduit. The cleaning fluid can then pass, like the lubricant, through these cavities into the interior of the handpieces and thus clean these. In other words, if desired, each lubricant spray connection also forms a cleaning fluidic connection, which connects by fluidic connection to the at least one bearing or gear part and/or the compressed air motor and thus allows a simple cleaning of the handpieces by machine.

A lubrication of the handpieces can then occur subsequently to the cleaning and a possibly still conducted steam sterilisation in particular to also lubricate parts of the handpieces susceptible to corrosion and assure an operation of the handpieces that is as low-friction as possible. Only a single spray head, which can be configured, for example, in one piece with or connected to a lubricant spray source in the form of a spray can, is necessary for lubrication.

What is claimed is:

1. A surgical handpiece for driving a surgical tool, with a housing, in which there are arranged a drive in the form of a compressed air motor and at least one bearing or gear part cooperating with the drive on the output side, further comprising at least one lubricant spray connection for coupling to a lubricant spray source, and which is in fluidic connection with at least one of the at least one bearing or gear part and the compressed air motor.

2. Surgical handpiece according to claim 1, further comprising at least one lubricant spray connection conduit for forming a fluidic connection between the at least one lubricant spray connection and the at least one bearing or gear part.

3. Surgical handpiece according to claim 1, wherein the housing comprises at least one seating for the at least one bearing or gear part, in which seating the at least one bearing or gear part is arranged, and wherein the at least one lubricant spray connection is in fluidic connection with the at least one seating.

4. Surgical handpiece according to claim 3, wherein the at least one lubricant spray connection conduit connects the at least one lubricant spray connection and the at least one seating to one another to form a fluidic connection.

5. Surgical handpiece according to claim 2, wherein the at least one lubricant spray connection conduit is configured in the form of a flexible tube- or pipe-type connection conduit or in the form of a connection channel arranged or configured on the housing.

6. Surgical handpiece according to claim 1, wherein the drive is arranged in the housing to be sealed in an airtight manner on the output side.

7. Surgical handpiece according to claim 1, comprising a single lubricant spray connection.

8. Surgical handpiece according to claim 1, wherein the at least one lubricant spray connection is arranged or configured at least one of in a housing wall of the housing and to project from the housing wall.

9. Surgical handpiece according to claim 1, wherein the at least one lubricant spray connection is arranged or configured on the output side of the drive.

10. Surgical handpiece according to claim 1, wherein the at least one lubricant spray connection is arranged or configured on the proximal side of the drive, and wherein the at least one lubricant spray connection conduit is arranged or configured laterally past the drive.

11. Surgical handpiece according to claim 1, wherein the at least one lubricant spray connection comprises a self-closing inlet valve.

12. Surgical handpiece according to claim 2, wherein the compressed air motor comprises a rotor shaft, and wherein the rotor shaft comprises the at least one lubricant spray connection conduit.

13. Surgical handpiece according to claim 12, wherein the rotor shaft is configured in the form of a hollow shaft, which defines a channel.

14. Surgical handpiece according to claim 13, wherein the channel forms the at least one lubricant spray connection conduit.

15. Surgical handpiece according to claim 13, wherein the at least one lubricant spray connection conduit is arranged in the channel and is configured to be fixed relative to the housing.

16. Surgical handpiece according to claim 12, wherein the at least one compressed air inlet is arranged or configured coaxially to the rotor shaft of the compressed air motor.

17. Surgical handpiece according to claim 12, wherein the at least one compressed air outlet is arranged or configured coaxially to the rotor shaft of the compressed air motor.

18. Surgical handpiece according to claim 2, wherein a closing element impermeable to compressed air in a closed position is arranged or configured in or on the at least one lubricant spray connection conduit.

19. Surgical handpiece according to claim 18, wherein the closing element is configured in the form of a self-closing valve, which is openable in a lubricating position.

20. Surgical handpiece according to claim 1, further comprising at least one lubricant spray connection closure for closing the at least one lubricant spray connection.

21. Surgical handpiece according to claim 20, wherein the at least one lubricant spray connection closure is arranged or configured on a compressed air coupling, which is couplable to the handpiece.

22. Surgical handpiece according to claim 16, further comprising at least one compressed air inlet and at least one compressed air outlet, which are in fluidic connection with the compressed air motor, and a self-closing exhaust air duct closing valve, which is closed in an operating position and is open in a lubricating position for forming a fluidic connection between an exhaust air duct connected to the at least one compressed air outlet and the compressed air motor and the at least one lubricant spray connection conduit.

23. Surgical handpiece according to claim 1, wherein the at least one bearing or gear part is configured in the form of a ball bearing, a sliding bearing, a needle bearing, a bearing shaft or a gear wheel.

24. Surgical handpiece according to claim 1, further comprising a surgical tool coupled to the handpiece on the distal side.

25. Surgical handpiece according to claim 24, wherein the tool is a drilling, milling, sawing, screwing or cutting tool.
